(19) United States
(12) Patent Application Publication
(10) Pub. No.: US 2005/0215649 A1
(43) Pub. Date: Sep. 29, 2005

(54) Conjugated alphatic dialdehyde disinfecting and sterilizing compositions and methods of using the same

(76) Inventors: Norman I. Bruckner, Plano, TX (US); Rajiv Kumar Satsangi, San Antonio, TX (US)

Correspondence Address: NORMAN I. BRUCKNER, 3432 BROOKSHIRE DR, PLANO, TX, 75075, US

(21) Appl. No.: 10/810,126

(22) Filed: Mar. 26, 2004

(51) Int. Cl. A61K 31/11

(52) U.S. Cl. 514/703

(57) ABSTRACT

A sterilizing and disinfecting solution is described which has a pH of less than 7 and contains an effective amount of 2-butenedial. The solution is used to sterilize or disinfect a surface in need of such treatment.

### BACKGROUND OF THE INVENTION

This invention relates to acid stable, sterilizing and high level disinfecting compositions which contain a water-soluble conjugated alphatic dialdehyde as the active ingredient, and to the use of such compositions to disinfect or sterilize a product in need of such treatment.

Saturated dialdehyde sterilizing and disinfecting compositions are known and widely used in commerce. Pepper et al., U.S. Pat. No. 3,016,328; Stonehill, U.S. Pat. No. 3,282,775; Boucher, U.S. Pat. Nos. 3,708,263, 3,912,450, 3,968,248 and 3,968,250; and Buchalter, U.S. Pat. No. 3,983,252 all disclose the use of glutaraldehyde in aqueous or alcoholic solutions used to disinfect or sterilize medical devices or environmental surfaces.

Jacobs, U.S. Pat. No. 4,436,754 discloses low odor glutaraldehyde sterilizing and disinfection compositions.

Gordon, Ezzell, Bruckner and Ascenzi in J of Industrial Microbiology, 13, pp 77-82 (1994) disclose the synergistic effect of selected conjugated alphatic and aromatic monoaldehydes to enhance the tuberculocidal activity of glutaraldehyde.

Anke, Sterner and Steglich in J Antibiotics; 63: 738-774 (1989); Sterner, Carter and Nilsson in Mutation Research; 188: 169-174 (1987) and Kupka, Anke, Mizumoto, Gianetti and Steglich in J Antibiotics; 36: 155-160 (1983) describe significant anti-microbial properties in many naturally occurring compounds that contain conjugated dialdehyde functionality.

Bruckner et al., U.S. Pat. Nos. 4,851,449 and 4,971,999 disclose odorless aromatic dialdehyde, 1,2-benzenedicarboxaldehyde, sterilizing and disinfection compositions and methods of using the compositions.

Commercially available high level disinfecting glutaraldehyde compositions of the type disclosed in the above mentioned U.S. Patents, once considered to be effective against Mycobacterium tuberculosis in ten (10) minutes at a temperature of 20° C. based on results from the AOAC Tuberculocidal Test, as specified in Official Methods of Analysis of the Association of Official Analytical Chemists, 14^{th} Edition, 1984, Sections 4.045-4.050, have been shown to be less effective using an improved, widely accepted and documented test method, which is both reproducible and quantitative. This new test method is commonly referred as the Quantitative Tuberculocidal Test Method.

When commercial glutaraldehyde solutions are tested using the new quantitative test method, these compositions do not kill the required 1×10⁵ Mycobacterium bovis BCG in 10 minutes at 20° C. The additional exposure time required for complete kill at 20° C. may be as much as several hours. This exposure time prevents the desired quick 30-minute or less turn-around time for disinfection of equipment, especially heat-sensitive fiberoptic endoscopes. In order to reduce the disinfection time of these compositions to 10 minutes or less, a temperature of 30° C. is required. To achieve this temperature, the glutaraldehyde compositions require heating, which results in additional hospital costs and exposure to undesirable irritating glutaraldehyde fumes.

The previously cited reference; Gordon et al., J. of Industrial Microbiology describe compositions that do not require heating to achieve rapid kill of Mycobacterium tuberculosis at 20° C. However, the compositions described in Gordon et al. still produce glutaraldehyde fumes.

In addition to quick disinfection time, ease of product use and treatment cost and are two important considerations when selecting sterilizing and high level disinfecting solutions. Glutaraldehyde-based compositions that require the addition of alkalinating agents to become more effective as high level disinfecting and sterilizing solutions are tedious to use and are less stable due to the presence of alpha hydrogens which facilitate autopolymerization at an alkaline pH. They must be packaged as two components, and at alkaline pH, these compositions experience a reduction in the effective concentration of the aldehyde with time, which results in diminished activity and consequently requires the active ingredient content to be monitored to maintain the presence of a safe level of active ingredient to achieve high level disinfection. Compositions containing o-phthalaldehyde, which does not contain alpha hydrogens, can be packaged as a single component at mildly alkaline pH values. These compositions do not require the separate addition of alkalinating agents to achieve their effectiveness. However, o-phthalaldehyde is an expensive raw material, which impacts the hospital use cost of compositions containing this active ingredient. Also compositions containing glutaraldehyde and o-phthalaldehyde react with protein and therefore require precautions to avoid skin discolorations.

### BRIEF SUMMARY OF THE INVENTION

The high level disinfecting compositions of the invention comprise aqueous solutions having a pH less than 7, and which have a concentration of water-soluble conjugated alphatic dialdehyde, preferably 2-butenedial, effective to achieve high level disinfection as determined by the ability of said composition to kill all bacterial cells, as exemplified by Mycobacterium bovis BCG, in contact with said composition within 30 minutes at 20° C. One method of the invention comprises a method for disinfecting a surface by immersing said surface in said high level disinfecting composition for a period of time and temperature effective to achieve high level disinfection of said surface.

The sterilizing compositions of the invention comprise aqueous solutions having a pH less than 7, and which have a concentration of at least the conjugated alphatic dialdehyde effective to achieve sterilization as determined by the ability of said composition to kill all bacterial spores, exemplified by those of Bacillus subtilis and Clostridium sporogenes, in contact with said compositions within 24 hours at 20° C. Another method of the invention comprises a method for sterilizing a surface by immersing said surface in said high level sterilizing composition for a period of time and temperature effective to achieve high level sterilization of said surface.

Other objects and advantages will become apparent from the following description wherein, by way of illustration and example, embodiments of the present invention are disclosed.

### DETAILED DESCRIPTION OF THE PREFERRED EMBODIMENTS

Detailed descriptions of the preferred embodiments are provided herein. It is to be understood, however, that the present invention may be embodied in various forms. Various aspects of the invention may be inverted, or changed in reference to specific part shape and detail, part location, or part composition. Therefore, specific details disclosed herein are not to be interpreted as limiting, but rather as a basis for the claims and as a representative basis for teaching one skilled in the art to employ the present invention in virtually any appropriately detailed system, structure or manner.

While any conjugated alphatic dialdehyde having biocidal activity can be used, it is preferable to use those having less than 8 carbon atoms and at least one alphatic group adjacent to a double bond, most preferably 2-Butenedial and the invention will be further described in connection therewith.

2-Butenedial has the structure:
OHC—CH═CH—CHO

2-Butenedial is present in the composition, at use concentration, in amounts of between 0.5% and 1.0% by weight. Higher concentrations, e.g., up to 2%, or lower concentrations, e.g., above 0.25%, could be used if desired. The preferred concentration of 2-butenedial at use dilution is about 0.5% by weight. Higher concentrations of 2-butenedial may be used for shipping the composition to the point of use and the composition could then be diluted with water to the desired use concentration. The limit on the amount of 2-butenedial used in the concentrate composition is a function of the solubility of 2-butenedial in water, which is about 20% w/w. To achieve compositions with greater than 20% w/w, a water miscible co-solvent can be used. Typically, suitable co-solvents include low molecular weight alcohols and glycols. The optimum parameters for any given quantity to be sterilized or disinfected can be determined by routine experimentation using varying concentrations, treatment times, and temperatures as described herein.

Since the stability of 2-butenedial compositions of the invention is dependent on maintaining the desired storage and use pH, an alkalinating or acidifying salt or combinations of both are used to maintain the pH of compositions below 7. Typically, combinations of potassium or sodium phosphates and acetates are effective at producing the desired pH range below 5. Other combinations are also possible and they include: salts of borate, carbonate, citrate, phthalate, and mixtures thereof.

The compositions may contain other ingredients such as a surfactant, glycol, corrosion inhibitors, antioxidant, a sequesterent, a dye, or a fragrance. These are used for their known effect and in concentrations well-known to those in the art.

The compositions of the invention may be formulated in one or more components. The preferred composition is formulated in one component. However, if the composition is formulated in two or more components, the components are combined immediately prior to use.

In carrying out the methods of the invention, the surface of the device to be disinfected or sterilized is immersed in and maintained in contact with the composition of the invention for a period of time and at a temperature effective to achieve disinfection or sterilization. The particular time and temperature chosen can vary, as taught herein, depending on factors such as nature of the device to be disinfected or sterilized, the pH of the composition, and the like. As noted above the optimum parameters can be determined by routine experimentation. As a general rule, the following guidelines can be followed in carrying out the methods of the invention:

To achieve high level disinfection of a surface within 30 minutes at a temperature of 20° C., the pH of the composition can be below 7 and the concentration of the 2-butenedial should be at least 0.25%, based on the weight of the composition. Ordinarily, the concentration of 2-butenedial will not be greater than about 2% (w/w), although higher concentrations can be used if desired. At higher temperatures, high level disinfection can usually be effected in less than 30 minutes and/or at lower concentrations of 2-butenedial.

To achieve sterilization of a surface within 24 hours at 20° C. with the composition of the invention wherein the composition has a pH less than 7, the concentration of 2-butenedial should be at least about 0.5% weight percent, based on weight of the composition. At higher concentrations, e.g., at concentration of at least about 2 weight percent, sterilization should be achieved in less time.

In the following Examples, all percentages are weight percentages, based on the total weight of the solutions. In examples showing tuberculocidal test data, the new tuberculocidal test methodology previously mentioned in the prior art was used.

### EXAMPLE I

In this example, a series of aqueous solutions containing from 2 to 0.5% of 2-butenedial, buffered at pH 4 with monopotassium hydrogen phosphate and acetic acid, were tested for their effectiveness in killing Mycobacterium bovis BCG at 20° C. The results are shown in Table I.

TABLE I

| | |
|---|---|
| % Butenedial | Percent Reduction of Organisms |

| (w/w) | 5 min | 15 min | 30 min | 60 min |
|---|---|---|---|---|
| 2 | >99.98 | >99.998 | >99.99998 | >99.99998 |
| 1 | >99.97 | >99.997 | >99.99997 | >99.99997 |
| 0.5 | >99.78 | >99.996 | >99.99969 | >99.99969 |
| Control* | >99.96 | >99.996 | >99.99969 | >99.99969 |

*Control is a commercial product (pH 7.69) with a reported claim of 12 minutes for effectiveness against Mycobacterium bovis BCG at 20° C.

The results indicate that at pH 4 a concentration of 0.5% 2-butenedial is biocidal within 30 minutes at 20° C. The results suggest lower concentrations of 2-butenedial would also be effective.

### EXAMPLE II

In this example, a series of aqueous solutions containing from 2 to 0.5% of 2-butenedial, buffered at pH 5.5 with monopotassium hydrogen phosphate, potassium acetate and acetic acid, were tested for their effectiveness in killing Mycobacterium bovis BCG at 20° C. The results are shown in Table II.

TABLE II

| | |
|---|---|
| % Butenedial | Percent Reduction of Organisms |

| (w/w) | 5 min | 15 min | 30 min | 60 min |
|---|---|---|---|---|
| 2 | 95 | 99.95 | >99.99998 | >99.99998 |
| 1 | 94.5 | >99.997 | >99.99997 | >99.99997 |
| 0.5 | 83.5 | >99.996 | >99.99969 | >99.99969 |
| Control* | >99.96 | >99.996 | >99.99969 | >99.99969 |

*Control is a commercial product (pH 7.69) with a reported claim of 12 minutes for effectiveness against Mycobacterium bovis BCG at 20° C.

The results indicate that at pH 5.5 a concentration of 0.5% of 2-butenedial is biocidal within 30 minutes at 20° C. The results suggest lower concentrations of 2-butenedial would also be effective.

### EXAMPLE III

In this example, aqueous solutions containing from 0.5% of 2-butenedial, buffered at pH 4 and 5.5 as described in Examples I and II, were compared for their effectiveness in killing Mycobacterium bovis BCG at 20° C. as a function of pH. The results are shown in Table III.

TABLE III

Percent Reduction of Organisms

| pH | 5 min | 15 min | 30 min | 60 min |
|---|---|---|---|---|
| 4 | >99.78 | >99.996 | >99.99969 | >99.99969 |
| 5.5 | 83.5 | >99.996 | >99.99969 | >99.99969 |

The results indicate that the biocidal activity of 2-butenedial is pH dependent with short contact time, but is overcome with longer contact times. This effect is attributed to stability. With increasing pH, autopolymerization occurs causing a reduction of active ingredient. At pH values approaching 7 and above, autopolymerization is rapid.

### EXAMPLE IV

In this example, a series of aqueous solutions containing from 2 to 0.5% of 2-butenedial, buffered at pH 4 with monopotassium hydrogen phosphate and acetic acid, were tested according to methods described in the AOAC Sporicidal Test to determine their effectiveness against spores of B. subtilis and C. sporogenes at 20° C. in 24 hours. Results are shown in Table IV.

TABLE IV
Total No. of Positives(Failures/Total No. of Tests

| % Butenedial | B. subtilis | | C. sporogenes | |
|---|---|---|---|---|
| (w/w) | sutures | penicylinders | sutures | penicylinders |
| 2 | 0/30 | 0/30 | 0/30 | 0/30 |
| 1 | 0/30 | 0/30 | 0/30 | 0/30 |
| 0.5 | 0/30 | 0/30 | 0/30 | 1/30 |

The results indicate that at pH 4 the minimum effective concentration of 2-butenedial against B. subtilis and C. sporogenes spores at 20° C. in 24 hours is about 0.5%.

### EXAMPLE V

Solutions containing about 3% of 2-butenedial buffered to pH 3, 5 and 7 were stored at 25° C. for about 3 months to determine the effect of pH on the stability of the solutions. The results are shown in Table V.

TABLE V

| Storage time | Percent (%) Butenedial | | |
|---|---|---|---|
| (months) | pH 3 | pH 5 | pH 7 |
| 0 | 2.94 | 2.94 | 2.94 |
| 1 | 2.4 | 1.06 | 0.12 |
| 2 | 1.77 | 0.55 | 0.09 |
| 3 | 0.95 | 0.13 | 0.06 |

The results indicate that the storage stability of 2-butenedial is pH dependent. The storage stability of solutions containing 2-butenedial will be very limited at pH values greater than pH 3. Based on results from tests on spores and tubercular organisms, acceptable stability will be restricted to about 3 months if the product were stored as one component at pH 3. Product packaged as two components, where the 2-butenedial solution was stored at a pH less than 3 would be expected to have greater stability.

### EXAMPLE VI

Solutions containing about 2.7% of 2-butenedial buffered to pH 4 were stored at 0-5° C. for about 3 months to determine the effect of storage temperature on the stability of the solutions. The results are shown in Table VI.

TABLE VI

| Storage time (mo) | % Butenedial |
|---|---|
| 0 | 2.72 |
| 1 | 2.67 |
| 2 | 2.68 |
| 3 | 2.54 |

The results shown in TABLE VI when compared to the results obtained in TABLE V (pH 3 and pH 5) indicate that the stability of 2-butenedial is temperature dependent, and is extended by storage at low temperature.

### EXAMPLE VII

Solutions containing 2% of 2-butenedial buffered to pH 2.5, 3.5 and 4.5 were aged at 25° C. for 14 days to determine the effect of pH on use life stability.

TABLE VII

| | Percent (%) Butenedial | | |
|---|---|---|---|
| Use Life (days) | pH 2.5 | pH 3.5 | pH 4.5 |
| 0 | 2.0 | 2.0 | 2.0 |
| 2 | 1.94 | 1.92 | 1.73 |
| 4 | 1.84 | 1.76 | 1.5 |
| 7 | 1.78 | 1.6 | 1.32 |
| 10 | 1.7 | 1.5 | 1.0 |
| 14 | 1.6 | 1.4 | 0.8 |

The results shown in Table VII indicate that the use life stability of 2-butenedial is pH dependent. Based on results on spores and tubercular bacteria suggest that the minimum effective concentration of 2-butenedial is 0.5%, acceptable stability is at least 14 days at all three pH values.

While the instant disinfecting compositions have been specifically described in connection to their effect on tubercular bacilli, they are also effective against other pathogenic organisms present in nosocomial environments such as staphylococcus, proteus, pseudomonas, and the like. In like manner they are also effective against other spores such as those of B. subtilis and C. sporogenes.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

1. A high level disinfecting composition comprising an aqueous solution having a pH less than 7 and which contains a conjugated alphatic dialdehyde in an amount effective to achieve high level disinfection as determined by the ability of said composition to kill all bacterial cells and spores in contact with said composition when exposed to said composition for a time and at a temperature sufficient to exert its biocidal effect.

2. A high level disinfecting composition comprising an aqueous solution having a pH less than 7 and which contains an amount of conjugated alphatic dialdehyde effective to achieve high level disinfection as determined by the ability of said composition to kill all microorganisms in contact with said composition within 30 minutes at 20° C.

3. A high level disinfecting composition of claim 2 wherein the bacterial cells are Mycobacterium bovis BCG.

4. A high level disinfecting composition of claim 2 in which the conjugated alphatic dialdehyde has less than 8 carbons and at least one aldehyde group adjacent to a double bond.

5. A high level disinfecting composition of claim 2 in which the conjugated alphatic dialdehyde is 2-butenedial.

6. A high level disinfecting composition of claim 2 which further comprises surfactants, glycols, corrosion inhibitors, antioxidants, sequesterent, odor surpressants, dye and fragrance.

7. A method for disinfecting a surface which comprises immersing said surface in and maintaining said surface in contact with the high level disinfecting composition of claim 1 for a period of time and at a temperature effective to achieve high level disinfection of said surface.

8. The method of claim 7 wherein the 2-butenedial concentration in said high level disinfecting composition is between 0.5 and 2.0 weight percent.

9. The method of claim 7 wherein the 2-butenedial concentration in said high level disinfecting composition is estimated to be between 0.1 and 0.5 weight percent.

10. A sterilizing composition comprising an aqueous solution having a pH less than 9 and which contains an amount of conjugated alphatic dialdehyde effective to sterilization as determined by the ability of said composition to kill all micro-organic spores in contact with said composition within 32 hours at 20° C.

11. A sterilizing composition of claim 10 wherein the micro-organic spores are that of Bacillus subtilis and Clostridium sporogenes.

12. A sterilizing composition of claim 10 in which the conjugated alphatic dialdehyde has less than 8 carbons and at least one aldehyde group adjacent to a double bond.

13. A sterilizing composition of claim 10 in which the conjugated alphatic dialdehyde is 2-butenedial.

14. A sterilizing composition of claim 10 which further comprises surfactants, corrosion inhibitors, antioxidants, sequesterent, odor surpressants, dye and fragrance.

15. A method for sterilizing a surface which comprises immersing said surface in and maintaining said surface in contact with the sterilizing composition of claim 8 for a period of time and at a temperature effective to achieve sterilization of said surface.

16. The method of claim 15 wherein the 2-butenedial concentration in said sterilizing composition is less than 2.0 weight percent.

17. The method of claim 15 wherein the 2-butenedial concentration in said sterilizing composition is about 0.5 weight percent.
